(19) United States
(12) Patent Application Publication
(10) Pub. No.: US 2014/0275793 A1
(43) Pub. Date: Sep. 18, 2014

(54) Minimally Invasive Retractor

(71) Applicant: John Song, Chicago, IL (US)

(72) Inventor: John Song, Chicago, IL (US)

(21) Appl. No.: 14/210,702

(22) Filed: Mar. 14, 2014

Related U.S. Application Data
(60) Provisional application No. 61/787,550, filed on Mar. 15, 2013.

(51) Int. Cl. A61B 17/02

(52) U.S. Cl. CPC A61B 17/0218
USPC 600/204

(57) ABSTRACT

A bone screw mounted retractor apparatus for use in minimally invasive surgery, preferably minimally invasive spine surgery, comprising a screw extension detachably attached to a bone screw, a mounting ring attached to the screw extension, a connection arm attached to the mounting dag, a retractor retaining ring attached to the connection arm, and a tubular retractor body retained by the retractor retaining ring, as well as methods of minimally invasive surgery using such an apparatus.

### CROSS-REFERENCES TO RELATED APPLICATIONS

This Application claims priority to and incorporates fully by reference U.S. Provisional Application 61/787,550 filed on Mar. 15, 2013.

### STATEMENT REGARDING FEDERALLY SPONSORED RESEARCH OR DEVELOPMENT

Not applicable

### BACKGROUND

One of the most challenging aspects of minimally invasive surgery is to be able to see or monitor the organ, bone, joint, or structure of concern without having to repeatedly clear away interfering matter, such as skin, blood, or nearby organs from the operative field. For example, the removal of an inflamed appendix requires the surgeon to be able to isolate this organ from its nearby surrounding tissues, such as the colon, sigmoid, ileum, jejunum, and ovaries. If this we ration were performed openly, through a large incision, then the surgeon has the option of using sterile towels to push the other intestines and organs away from the inflamed appendix, thereby creating a clear visual field for the safe removal of the organ. In a minimally invasive procedure, the surgeon does not have the option of using a sterile towel.

Typically, the visualized operative field in a minimally invasive procedure is narrow. Therefore, the surgeon must frequently “clear the field” by pushing interfering matter away as it covers up the structure of concern, or try to utilize the tilt function of the operating room table to encourage the interfering matter to fall away from the operative field. These methods of clearing the operative field are inefficient and potentially dangerous for the patient

One solution known to the art is the use of as retractor adapted for minimally invasive procedures. While there has been considerable development of retractors and retractor systems for minimally invasive procedures, many of these developments are based on retractors optimized for open procedures and are predominantly table-mounted. Retractors known to the art thus tend to he cumbersome and are not well adapted for use in conjunction with the small incisions standard in minimally invasive procedures.

Standard hand-held surgical retractors fur open surgery are well known in the art and can be modified to it the contours of minimally invasive incisions, but such retractors require manual manipulation to maintain desired placement, thereby occupying one hand of the physician or requiring another person to assist the physician during the procedure. Retractors known to the art suffer additional problems. For example, retractors known to the art frequently requiring re-positioning if they dislodge, can obstruct the physician's view, and can otherwise interfere with access to the surgical site.

In recent years, minimally invasive surgical approaches have been applied to orthopedic surgery and more recently to spine surgery, such as instrumented fusions involving one or more vertebral bodies. Unlike minimally invasive procedures such as arthroscopic knee surgery or appendix removal, where the affected area is contained within a small region of the body, spinal fusion surgery typically encompasses a considerably larger region of the patient's body. In addition, while minimally invasive procedures on other portions of the body such as the abdomen or the knee permit the introduction of fluid for distending tissue and creating working space for the surgeon, the spine does not involve a capsule or space that can be so distended, instead involving multiple layers of soft tissue, bone, ligaments, and nerves. For these reasons, the idea of perforating a minimally invasive procedure on the spine has only recently been approached.

In as traditional spine fusion at least two vertebral bodies are rigidly connected using screws implanted into the respective vertebral bodies with as solid metal rod spanning the distance between the screws. This procedure is more complicated in a minimally invasive approach. While the insertion of bone screws, such as pedicle or facet screws, can be accomplished straightforwardly through a minimally invasive incision, the introduction of a length of rod is more difficult with a minimally invasive incision, which provides limited access and visibility. A minimally invasive incision is typically approximately 1 centimeter long. A single level fusion procedure performed traditionally, however, may require the introduction of a rod of 40 mm in length, and a multilevel fusion may require the introduction of a rod several inches long. Due to the relatively large length of the rod and the relatively small length of the incision, it is important in minimally invasive spine surgery that the minimal incision be maintained in an open and accessible condition. Retractors known to the art do not satisfactorily perform this function without unduly interfering with the access, vision, and working space of the surgeon.

It is known to the an to attempt as minimally invasive spinal fusion by making minimally invasive incisions, then placing an interbody cage into the disc space with the assistance of a table-mounted tubular retractor. The retractor is then removed and bone screws are placed. However, retractors known to the art are bulky and, their table-mounting apparatus interferes with the surgeon's movement during the fusion procedure. Additionally, table-mounted retractors known to the art do not allow for ready movement of the distal end of the retractor during the fusion procedure.

Embodiments of the present invention are directed to a novel retractor apparatus for use in minimally invasive surgery and methods of using the same. The apparatus described herein may be made of any suitable biocompatible material, or materials, as will be appreciated by one skilled in the art.

Spinal surgery commonly involves the use or placement of one screws, often pedicle screws, into one or more vertebral bodies. Embodiments of the present invention include a bone screw detachably, and optionally polyaxially, attached to a screw extension. A mounting ring is slidably connected to the screw extension, and may optionally be locked into place along the screw extension through use of a set screw or clamp. A connection arm is attached at one end to the mounting ring and at the other end to a retractor retaining ring with an interior circumference suitable to retain a tubular retractor of desired size. The interior surface of the retractor retaining ring is optionally threaded or grooved. A tubular retractor is retained by the retractor retaining ring. The tubular retractor is optionally threaded or grooved at least at its distal end, is optionally retained by a threaded connection with the retractor retaining ring, and can optionally be height-adjusted by turning or twisted the tubular retractor within the retaining ring.

In embodiments of the present invention, the tubular retractor can be adjusted by one or more of: rotation of the screw extension, polyaxial adjustment of the screw extension, rotation of the mourning ring about the screw extension, elevation or depression of the mounting ring along the screw extension, rotation of the connection arm with respect to the mounting ring, by rotation of the retractor retaining ring with respect to the connection arm, or by adjusting the height of the tubular retractor within the retractor retaining ring. Embodiments of the present invention thus allow a retractor to be adjusted into virtually any orientation or position without the interference of cumbersome table-mounting apparatus and without a loss of stability by the retractor itself.

The retractor apparatus of the present invention may he used in a novel method of performing surgery. In general, this method comprises the steps of installing a bone screw in a bone, serially dilating soft tissues to be retracted, detachably attaching a screw extension to the bone screw, mounting a retractor to the screw extension, adjusting the retractor to the desired height and orientation, and retracting tissue.

In the Summary above and in the Description, and the claims below, and in the accompanying drawings, reference is made to particular features of the apparatus and of the method. It is to be understood that the disclosure of the invention in this specification includes all possible combinations of such particular features. Reference is further made to various steps of methods. It is to be understood that these steps need not be performed in the order listed.

### BRIEF DESCRIPTION OF THE DRAWINGS

These and other features, aspects, and advantages of the present invention will become better understood with regard to the following description and accompanying drawings, where:

FIG. 1 shows an end-on view of an embodiment of as retractor apparatus mounted to a bone screw;

FIG. 2 shows a partially exploded view of one embodiment of a retractor apparatus;

FIG. 3 shows an end-on view of an embodiment of a retractor apparatus mounted to a bone screw installed in a posterior approach minimally invasive spine procedure;

FIG. 4 shows a side view of an embodiment of a retractor apparatus mounted to a one screw installed in a posterior approach minimally invasive spine procedure;

FIG. 5 shows an end-on view of an embodiment of a retractor apparatus mounted to a bone screw installed in a lateral approach minimally invasive spine procedure;

FIG. 6 shows a side view of an embodiment of a retractor apparatus mounted to a bone screw installed in a lateral approach minimally invasive spine procedure;

### DESCRIPTION

Embodiments of the present invention are directed to a retractor apparatus and methods of surgery using a retractor apparatus. In preferred embodiments, the retractor apparatus is used in connection with the performance of minimally invasive spine surgery, and, most preferably, minimally invasive spine fusion. Embodiments of the present apparatus and method can be used in virtually any method of minimally invasive spine surgery, including, by way of illustration, posterior approach procedures, lateral approach procedures, and anterior approach procedures.

A “bone screw” is, as will be recognized, by one skilled in the art, a screw adapted to be fixed to or installed in bone, and preferably to a vertebra. A “bone screw” herein comprises a screw adapted for fixation to bone, and also comprises a screw head, wherein the screw head is adapted for connection with a screw extension [3], as described elsewhere herein. Preferably, the bone screw is a pedicle screw or a facet screw. Other forms and types of screws may be used within the scope of this invention, as will be apparent to one skilled in the art.

Preferably, the bone screw head comprises in part a collar [5], said collar comprises in part an aperture [7], and said collar 151 further comprises a set screw [9] such that when a screw extension [3] is placed in or through said aperture [7] and said set screw 91 is tightened, said screw extension and said bone screw head become rigidly attached. As will be appreciated by one skilled in the art, a rigid attachment achieved in this manner can be separated by loosening the set screw [9]. Said aperture can pass all the way through the collar [5], or may optionally he a “blind” aperture haying an opening only on one side.

A “screw extension” [3] herein is an elongated member adapted for detachable connection to a bone screw. Preferably, the screw extension [3] comprises a primary elongated member [11] and a joint [15] permitting polyaxial rotation of the primary elongated member [11]. Optionally, the screw extension [3] may flintier comprise a second elongated member connected to the joint [15] opposite the primary elongated member [11]. In preferred embodiments, the joint [15] is located near the distal end [17] of the. screw extension [3]. in a most preferred embodiment, the joint [15] is located substantially at the distal end [17] of the screw extension [3].

A “mounting ring” [19] herein is a ring adapted for slidable connection to the primary elongated member [11] of the screw extension [3]. Such slidable connection can be accomplished by a variety of mechanical connection means, as will be recognized by one skilled in the art. Preferably, the mounting ring [19] can be adjusted to one or more locations along the length of the screw extension [3], most preferably along the length of the primary elongated member [11]. Most preferably, the mounting ring [19] cut be releasably locked at one or more locations along the screw extension [3]. Such releasable locking can be accomplished by a variety of locking mechanisms, such as set screws or clamps, as will be recognized by one skilled in the art.

A “connection arm” [21] herein is attached at one end to the mounting ring [19] and at the other end to a retractor retaining ring [23]. A connection arm [21] comprises an elongated member, rod, bar, plate, or other structure forming a physical connection between the mounting ring [19] and the retractor retaining ring [23]. Preferably, the attachment between the connection arm [21] and one or more of the mounting ring [19] and retractor retaining ring [23] is rotatable. Most preferably, the attachment between the connection arm [41] and one or more of the mounting ring [19] and retractor retaining ring [23] is rotatable such that the retractor retaining ring [23] remains in an orientation substantially perpendicular to vector defined substantially by the primary elongated member [11].

A “retractor retaining ring” [23] herein is a retaining ring with an interior circumference suitable to surround a tubular retractor [25] of desired size, as will be appreciated by one skilled in the art. Preferably, the retractor retaining ring [23] comprises one or more of threads and grooves on its interior surface adapted to engage with corresponding threads or grooves on the exterior surface of the tubular retractor [25].

A “tubular retractor” herein is to tubular retractor body. As will be appreciated by one skilled in the art, a large variety of existing tubular retractors could be used or modified to be used in connection with the apparatus and methods described herein. In embodiments herein, the tubular retractor [25] detachably attaches to the retractor retaining ring [23] through one or more of grooves or threads on the exterior surface of the tubular retractor [25] and corresponding grooves or threads on the interior surface of the retractor retaining ring [23]. Preferably, the tubular retractor [25] comprises one or more of grooves and threads on its exterior surface adapted to engage with corresponding threads or grooves on the interior surface of the retractor retaining ring [23]. Preferably, the grooves or threads of the tubular retractor [25] and retractor retaining ring [23] are configured such that the height of the tubular retractor [25] can be altered by turning or twisting the tubular retractor [25] when the tubular retractor is threadably attached to the retractor retaining ring [23].

A retractor apparatus as described herein can be used in methods of performing surgery. Such methods comprise the steps of placing a bone screw in a bone, serially dilating soft tissue to be retracted, detachably attaching a retractor apparatus to the bone screw, adjusting the retractor body of the retractor apparatus to the desired height and orientation, and using the retractor to retract dilated tissue. Preferably, the retractor apparatus used in connection with these methods comprises a screw extension detachably and polyaxially attached to a bone screw, a mounting ring [19] slidably connected to the screw extension [3], to connection arm [21] adjustably attached at one end to the mounting ring [19] and at the other end to a retractor retaining ring [23], and a tubular retractor [25] threadably attached to the retractor retaining ring [23]. Embodiments of the method taught here in can be utilized in minimally invasive spine procedures involving virtually any approach, including, by way of illustration, a posterior approach, an anterior approach, or lateral approach.

Although the present invention has been described in considerable detail with reference to certain preferred versions thereof, other versions are possible. For example, other bone screws, materials, retractor types, or adjusting or locking means, may be used than those described in detail. Similarly, other placements of embodiments of the invention may be employed than those shown in detail and the invention may be used to retract tissue at locations on the body other than those described herein. Further, the steps of methods disclosed herein need not be performed in the order described in detail herein, and additional steps may be added within the spirit of the scope of the invention. Therefore, the spirit and scope of the claims should not be limited to the description of the preferred versions described herein.

What is claimed is:

1. A retractor apparatus for use in minimally invasive surgery, said retractor apparatus comprising
a. a hone screw;
b. it screw extension detachably attached to said bone screw;
c. a mounting ring attached to said screw extension;
d. a connection arm attached to said mounting ring;
e. a retractor retaining ring attached to said connection arm; and
f. a tubular retractor body retained by said retractor retaining ring.

2. The apparatus of claim 1, wherein said screw extension comprises a primary elongated member and a joint.

3. The apparatus of claim 2, wherein said primary elongated member is polyaxially adjustable with respect to said bone screw.

4. The apparatus of claim 3, wherein said screw extension further comprises a secondary elongated member.

5. The apparatus of claim 3, wherein said mounting ring is slidably attached to said screw extension.

6. The apparatus of claim 5, wherein said mounting ring is lockably attached to said screw extension.

7. The apparatus of claim 3, wherein said connection arm is rotatably attached to one or more of said mounting ring and said retractor retaining ring.

8. The apparatus of claim 3, wherein said retractor retaining ring is substantially perpendicular to the vector substantially defined by the primary elongated member.

9. The apparatus of claim 3, wherein said retractor retaining, ring further comprises one or more of grooves and threads on its interior surface.

10. The apparatus of claim 9 wherein said tubular retractor comprises one or more of grooves and threads on its exterior surface.

11. The apparatus of claim 10, wherein said one or more of grooves and threads on the exterior surface of said tubular retractor are located substantially at the distal end of said tubular retractor.

12. The apparatus of claim 10, wherein said one or more of grooves and threads on the interior surface of said retractor retaining ring are adapted for threaded connection with said one of more of grooves and threads on the exterior of said tubular retractor.

13. The apparatus of claim 12, wherein the height of said tubular retractor is adjusted by turning said tubular retractor when said tubular retractor is within said retractor retaining ring.

14. A method for performing minimally invasive surgery, said method comprising the steps of:
a. Placing a bone screw in a bone;
b. Serially dilating soil tissue to be retracted;
c. Attaching a retractor apparatus to said bone screw, said retractor apparatus comprising a screw extension detachably attachable to said bone screw; a mounting ring attached to said screw extension; a connection arm attached to said mounting ring; a retractor retaining ring attached to said connection arm; and a tubular retractor body retained by said retractor retaining ring;
d. Adjusting said retractor apparatus to the desired height and orientation; and
e. Using said retractor apparatus to retract tissue.

15. The method of claim 14, wherein said screw extension comprises a primary elongated member and a joint and said primary elongated member is polyaxially adjustable with respect to said bone screw.

16. The method of claim 15, wherein said mounting ring is one or more of slidably attached and lockably attached to said screw extension.

17. The method of claim 15, wherein said connection arm is rotatably attached to one or more of said mounting ring and said retractor retaining ring.

18. The method of claim 15, wherein said retractor retaining ring is substantially perpendicular to the vector substantially defined by the primary elongated member.

19. The method of claim 15, wherein said retractor retaining ring further comprises one or more of grooves and threads on its interior surface adapted for threaded connection with said tubular retractor.

20. The method of claim 19, wherein said tubular retractor further comprises one or more of grooves and threads on its exterior surface adapted for threaded connection with said retractor retaining ring.

21. The method of claim 20, wherein said one or more of grooves and threads on the exterior surface of said tubular retractor are located substantially at the distal end of said tubular retractor.

22. The method of claim 15, wherein said minimally invasive surgery comprises spine surgery employing at least one of an anterior approach, a posterior approach, and a lateral approach.
